(19) United States
(12) Patent Application Publication
(10) Pub. No.: US 2004/0262240 A1
(43) Pub. Date: Dec. 30, 2004

(54) Heating, ventilation or air conditioning water purifiers

(76) Inventor: Simon Oke, Wingfield (AU)

Correspondence Address: FINNEGAN, HENDERSON, FARABOW, GARRETT & DUNNER, LLP, 1300 I STREET, NW, WASHINGTON, DC, 20005, US

(21) Appl. No.: 10/482,663

(22) PCT Filed: Jul. 4, 2002
(86) PCT No.: PCT/AU02/00881

(30) Foreign Application Priority Data
Jul. 6, 2001 (AU) PR 6207

(51) Int. Cl. B01J 19/08

(52) U.S. Cl. 210/758; 210/760; 422/186.07

(57) ABSTRACT

A method and apparatus for the continuous or regular cleaning and purification of water in HVAC systerns, such as Evaporative Coolers, Cooling Towers and Hot/Warm Water Systems. Oxidants and oxidant radicals are produced electrically in a stream of air and the resultant gas is injected into a stream of water which flows through the HVAC system and where further oxidants may be generated in this downstream flow of water.

### BACKGROUND OF THE INVENTION

HVAC Water Purifiers are products which control water quality in Heating, Ventilation or Air Conditioning (HVAC) systems. This includes Evaporative Coolers, Cooling Towers and Hot/Warm Water Systems. In these three applications, a flow of water is central to the process. Pollutants such as legionella bacteria and scale occur in these applications.

Evaporative Coolers are products which use water to cool air. Thus water and air are the working fluids, and the cooled air is distributed for ventilation. Evaporative Coolers also increase the humidity of the air. They are used in houses and commercial premises where they are often located on the roof of the building. In many applications they are connected to ducting, downstream, to supply the cooled air to various rooms in the building. But they do not include return ducting and are therefore not a recirculating refrigerative air conditioning system (and such refrigerative systems do not utilise a flow of water). They are common in regions where the ambient climate is relatively hot and dry, such as Australia, The Middle East, Southern USA, India and Southern Europe. In such regions they compete against refrigerative air conditioning systems, including ducted recirculating systems and split systems.

An Evaporative Cooler with an HVAC Water Purifier is shown conceptually in FIG. 1. A typical Evaporative Cooler works as follows. Water regularly recirculates through pipework within the Cooler itself. The water leaves the basin 3 through pipework at 1 and is pumped by a water pump 2, from the basin to a position where it enters the top of the Cooler at 4. It then falls by gravity to the basin again, through a flow of air. Make-up water 5 is supplied to the Evaporative Cooler to replace water which is evaporated. Some water exits the system 6, known as “bleed or dump or purge or blow-down water” so as to remove salts and other pollutants. The sides of the Cooler comprise permeable pads 7. The water trickles downwards through these pads. Relatively warm air is sucked from the outside ambient environment, through the pads, to the inside of the Cooler. The air is moved by a large fan 8 which is located inside the Cooler. As the air passes through the moist pads, an evaporative process takes place and the air temperature reduces and the air humidity level Increases. The relatively cool air, is then distributed by the same fan, to the inside of the building. Often this occurs through a supply duct system, but sometimes the air is blown directly into a space below the Cooler. Evaporative Coolers often also contain various control devices and enhanced features. In the building itself, it is necessary that some windows or doors or similar are left open, so that the humidified cool air, supplied by the Evaporative Cooler, can escape to the ambient environment again.

The HVAC Water Purifier 9 is fastened or hung in any position, preferably inside the Evaporative Cooler so that cool air flows over it, to remove heat build-up from the Purifier device. It may be located as shown in FIG. 1 or it may be located at a lower location in a control bay which houses the Coolers water pump and electrics. The Purifier has an inlet tube 10 which delivers air to it, preferably from a location where the air is relatively dry, such as the outside of the product. The Purifier also has an outlet tube 11 to deliver the oxidants to the water, preferably in the basin, where it is dissolved in the water by using a porous diffuser 12, or alternatively a venturi and water pump arrangement. The Purifier is connected to an electrical supply, such as the terminals of the Coolers water pump or fan.

Cooling Towers (also known as cooling water systems or water cooling systems) are products which use air to cool water. They are closely related to Evaporative Coolers and infact are reverse evaporative coolers. Thus water and air are the working fluids and the cooled water is distributed for various purposes such as the cooling of ventilation equipment inside a building, They are used in commercial premises where they are often located on the roof of the building. They are also used in industry where water is used to cool heat exchangers, machines, processes or products.

A Cooling Tower with an HVAC Water Purifier is shown conceptually in FIG. 2. Its components are similar to the Evaporative Cooler as follows. Make-up water 5 is supplied to the Cooling Tower to replace water which is evaporated. Some water exits the system 6, known as “bleed or dump or purge or blow-down water” so as to remove salts and other pollutants. The vertical structure of the Cooling Tower comprises permeable pads or racks or slats 7. Relatively warm “process water” enters through pipework at the top of the Cooling Tower 4 and then trickles downwards through these pads into the basin 3. Air is sucked from the outside ambient environment, through the pads, to the inside of the Cooling Tower. The air is moved by a large fan 8 which is located inside the Cooling Tower. As the water passes through the aerated pads, heat transfer takes place and the water temperature reduces. The relatively cool “process waters” then leaves the basin through pipework 1. It is distributed by a water pump 2, through pipework to perform the required work of cooling ventilation systems etc. Cooling Towers also contain various control devices and enhanced features.

The HVAC Water Purifier 9 is located in any position, such as outside the Cooling Tower. A relatively small diameter dedicated pipe 13 is installed which recirculates water around the basin to the Purifier. The Purifier has an outlet tube 11 to deliver and dissolve the oxidants in the water, typically by using a venturi 14 and small water pump arrangement 15. Alternatively the venturi and small water pump are an integral part of the Water Purifier. The treated water is then returned to the pipe 13 where it mixes with the water and then returns to the basin. The Purifier is connected to an electrical supply, preferably with a timer so that it is “on” before Cooling Tower operation commences.

Hot/Warm Water Systems are products which use gas or oil or electricity or sunlight to heat water. They can also use refrigerative or other fluids in heat pump devices to heat water. Thus water is the primary working fluid, and the heated water is distributed for usage to taps and other exit points. They are used in houses and commercial premises. Conventionally, most residential and commercial premises use hot water systems. However some commercial premises such as nursing homes and hospitals use dedicated recirculating warm water systems, so as to avoid the risk of scalding the user at the tap or exit point. More recently, many residential premises now utilise Tempered Hot Water Systems, where hot water is created, then mixed with some cold water to create warm water before being distributed to taps and exit points. The term “Hot/Warm Water System” is used to refer to the above three types of systems.

A Hot/Warm Water System with an HVAC Water Purifier is shown conceptually in FIG. 3. The example shown is a Tempered Hot Water System. Relatively cold water is supplied to the System at 16. It then enters a heating device such as a tank or array of pipework 17 where it is heated by various means such as sunlight, electricity, gas or oil 18. For example the hot water temperature may be 75 degrees Celsius. The hot water then leaves the heating device through pipework 19, either “on-demand” when taps are opened, or through a recirculating pipe system. The hot water then enters a mixing device 20 such as a mixing valve where it is mixed with cold water 21 to create warm water 22. For example the warm water temperature may be 40 degrees Celsius. The warm water then exits at taps and other locations, where of course the user may further mix the warm water and cold water to a desired temperature. At these exit points, the water passes through air, and thus water aerosols may form, where water droplets are suspended in air. Hot/Warm Water Systems may also contain various control devices and enhanced features,

The HVAC Water Purifier 9 is fastened or hung in any position, preferably near the mixing valve. The Purifier has an inlet tube 10 which delivers air to it. The Purifier also has an outlet tube 11 to deliver the oxidants to the water in the pipework, where it is dissolved in the water by using a venturi or other contacting device 14. The Purifier is preferably connected so that it operates whenever water is flowing through the mixing valve. Therefore it may be connected to an electric solenoid in the mixing valve, or to a flow switch located in or adjacent to the pipework so that an electric signal is sent to the Purifier.

The art concerns water quality control devices in HVAC systems, where water is always present as a working fluid and is cooled or heated, and air is also present as a working fluid or is present at the exit point from the system. The air which is passed through the water (in the case of Evaporative Coolers ard Cooling Towers), may of course be Inhaled by people in a cooled space or by people who are present in the outside environment. The air at a system exit point (in the case of Hot/Warm Water Systems) may be inhaled by users in the form of a water/air aerosol or of course the water may be ingested. Therefore the quality of the air and the water are important for reasons of human health and safety, and they are also important for reasons regarding effective operation of the HVAC devices:

A wide range of micro-organisms can breed in the water and pads, including bacteria, viruses, protozoa, algae and fungi. The micro-organisms include legionella bacteria which can cause Legionnaires Disease and Cryptosporidium and Giardia protozoa which can also be lethal. Water temperatures in Cooling Towers, Evaporative Coolers and in the warm water component of Hot/Warm Water Systems, all allow the growth of Legionella bacteria and dangerous protozoa.

If salts exist in the feed water, then the concentrations of those salts may build up over time in the System. The salts may deposit on surfaces (both wetted surfaces and dry surfaces downstream), thus forming mineral scale or lime, This scale can cause moving parts to foul and can cause corrosion. This reduces the life of components, increases the need for servicing, increases failure rates, causes aesthetic problems such as the formation of white stains, etc. It can also reduce the cooling efficiency of pads, and increase usage of electricity or fuel,

Organic scale may also buildup in pipes, basins and vessels. This includes bio-film, bio-slime, fungus, algae, bacteria and protozoa, which adhere to surfaces. These substances must be removed periodically, otherwise the surfaces becomes unacceptably dirty and equipment may also become blocked.

Unpleasant odours can occur, associated with generally poor water hygiene. This can affect occupants as well as neighbourng residences.

The colour of the water can become unpleasant. This is a problem when occupants sight the water in the system or in the case of bleed water which leaves a Cooler or in the case of ingested water with Hot/Warrn Water Systems.

The water can cause unacceptable rates of corrosion in the System.

Water usage can be excessive, causing “environmentally unfriendly” conditions due to poor water conservation and excessive waste water.

Running costs can be high due to high water usage, and the need for regular maintenance.

Various methods are currently used in HVAC Systems to control water quality, including purge water and chemicals:

Purge water may also be called bleed, dump or blow-down water, depending on the method used. Water may be continuously or semi-continuously bled or discharged as an overflow from the System. In this way, unacceptable concentrations of salts or microbes or odours, exit the system on a regular basis. For example, a residential Evaporative Cooler may operate with the following flow rates: 90 litres/hour of make-up water (from mains supply), 50 litres/hour of evaporated water leaving the system, and 40 litres/hour of bleed water leaving the system. Alternatively, water may be periodically dumped from the System, so that there is then no water in the system, either for a temporary moment, or during an “off-season”. For example, a residential Evaporative Cooler may dump once per day when the Cooler is turned off by the householder, emptying the basin of 20 litres of water. Other hybrid methods of purge also exist, including methods that are triggered by water salinity measurements.

Purge water leads to expensive water usage and wastage. For example, if a single residential Evaporative Cooler uses 40 litres/hour of bleed water, then at 10% utilisation over the year, the water wasted is approximately 40,000 litres per house. The ability of purge water to effectively control salts, micro-organisms, odour, colour and corrosion —is limited. Typically these problems still occur to some extent, at undesirable levels.

Many HVAC systems are cleaned and purified periodically. The word “clean” may be used to refer to the removal of deposits and debris and scale from the inside of the surfaces. The word “purify” may be used rather differently to refer to the killing of microbes on the inside of the surfaces. Therefore it is necessary to achieve both objectives —to clean and to purify. Evaporative Coolers and Cooling Towers and commercial Hot/Warm Water Systems, for example may be cleaned yearly. Chemicals as well as mechanical or manual methods are the common methods used.

Various products and special equipment are also available for continuous “in product” cleaning and purifying. Most use liquid chemicals which are fed into a chemical dosing pump device such as a centrifugal pump. Or chemicals enter the system in the form of chlorine in mains water. A “non-chemical” product is ultraviolet radiation, where a UV device may be located in the water pipework.

Chemicals are consumables and therefore they need to be frequently purchased, transported and stored and dispensed. This creates significant on-going purchasing and logistics costs. The chemicals are hazardous in nature. This creates occupational health and safety problems, during transport, storage and handling. Handling may include the need to transport the chemicals to the top of a roof. The chemicals and pollutants, following cleaning, create a disposal problem. Typically they should not be run to the drain or sewer, because they are toxic, If they are not, then alternative the law, Some chemicals do not clean efficiently or purify efficiently, especially in the case of protozoa such as Cryptosporidium and Giardia, and also in the case of Legionella bacteria. Some chemicals are excessively corrosive.

Ultraviolet radiation can purify water which passes through its radiation field if the turbidity of the water is such that the radiation can penetrate. However Ultraviolet does not clean surfaces downstream or purify water downstream, as it does not create any lasting agents which enter the flow of water.

### OBJECTS

It is an object of this invention to overcome one or more of the above problems associated with the control of water quality in HVAC systems.

A further object of the invention is to provide a system for controlling the quality of water and of wetted and dry surfaces in HVAC systems in which consumables are not required to be purchased and in which no polluting or hazardous materials are used.

### BRIEF STATEMENT OF THE INVENTION

Thus there is provided according to the invention a method of cleaning and purifying water and surfaces in HVAC systems including the steps of electrically producing oxidants by passing air through an oxidising chamber such as a corona discharge chamber, mixing the oxidants with the flow of water whereby the oxidants in the water passing through the HVAC system cause contaminants in the system, including scale and microorganisms, to be removed, oxidised, killed or flocculated and filtered.

Also there is provided according to the invention a method of cleaning and purifying HVAC systems, including the steps of producing ozone and/or hydroxyl radicals in the water which flows through the HVAC system to react with and remove contaminants in the water and on surfaces.

Additionally there is provided according to the invention a method of cleaning and purifying HVAC systems including the steps of passing air which contains oxygen and water vapour through an oxidising chamber to produce one or more oxidants in the form of ozone, hydrogen peroxide, hydroxyl radicals, hydroxyl ions, atomic oxygen, and atomic oxygen ions and injecting and mixing the oxidants in the flow of water through the HVAC system.

There is also provided apparatus for cleaning and purifying HVAC systems, said apparatus including an air inlet, an oxidant or ozone generator having an inlet connected to the air inlet, and an outlet connected to a passage between the water inlet and outlet whereby the products from the oxidant or ozone generator are passed into and mixed with the water to clean and purify the HVAC system.

There is also provided apparatus for cleaning and purifying HVAC systems, said apparatus including means of micro-flocculating salts in the water, producing a motive force by bubbling air through a friction tube in water, and passing this flocculated material and water through a water filter, thus capturing the salts.

### BRIEF DESCRIPTION OF THE DRAWINGS

In order to more fully describe the invention reference is now made to the accompanying drawings in which;

FIG. 1 is an example of the water purifier located in an evaporative cooler,

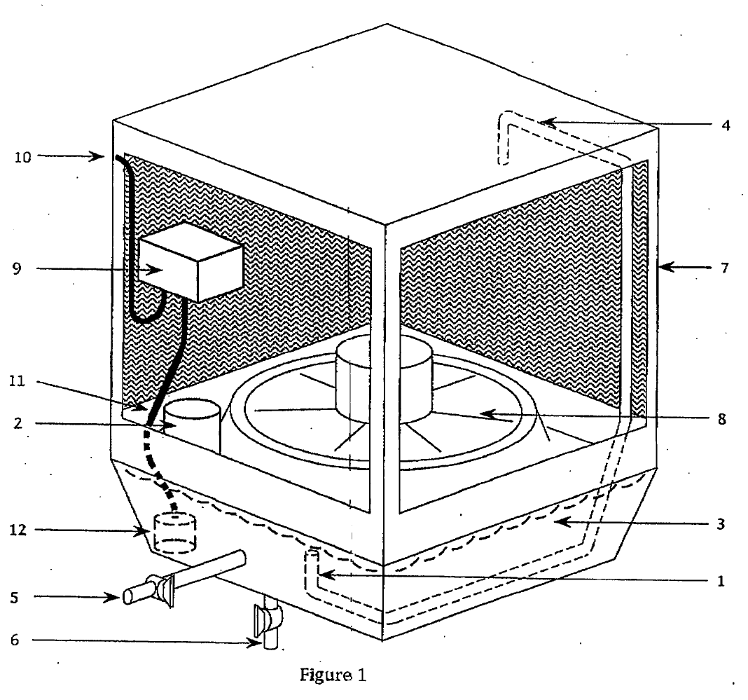

FIG. 2 is an example of the water purifier located in a cooling tower,

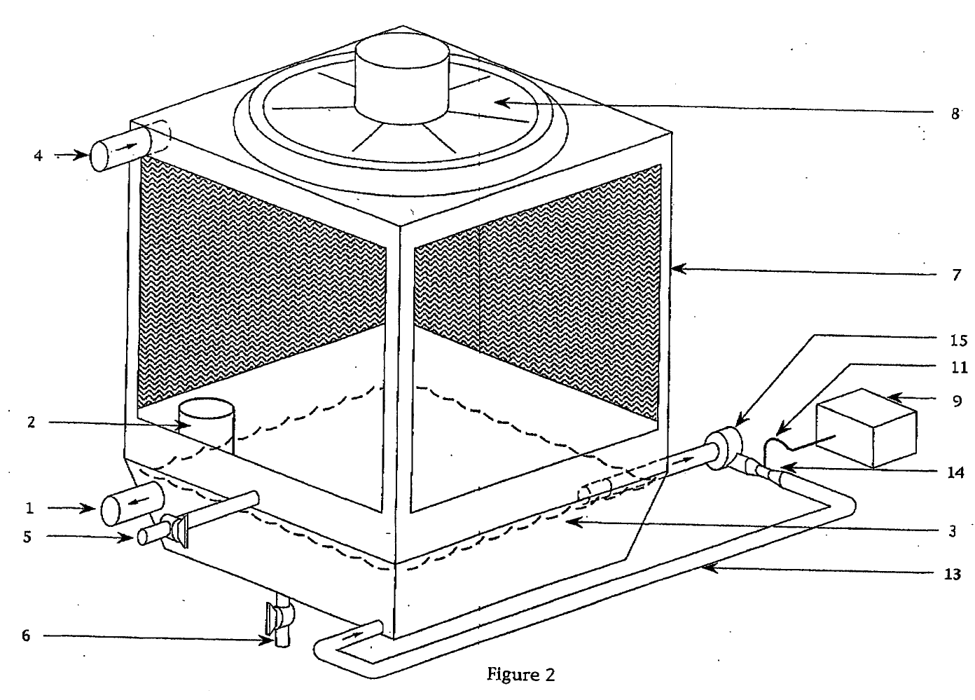

FIG. 3 is an example of the water purifier located in a hot/warm water system,

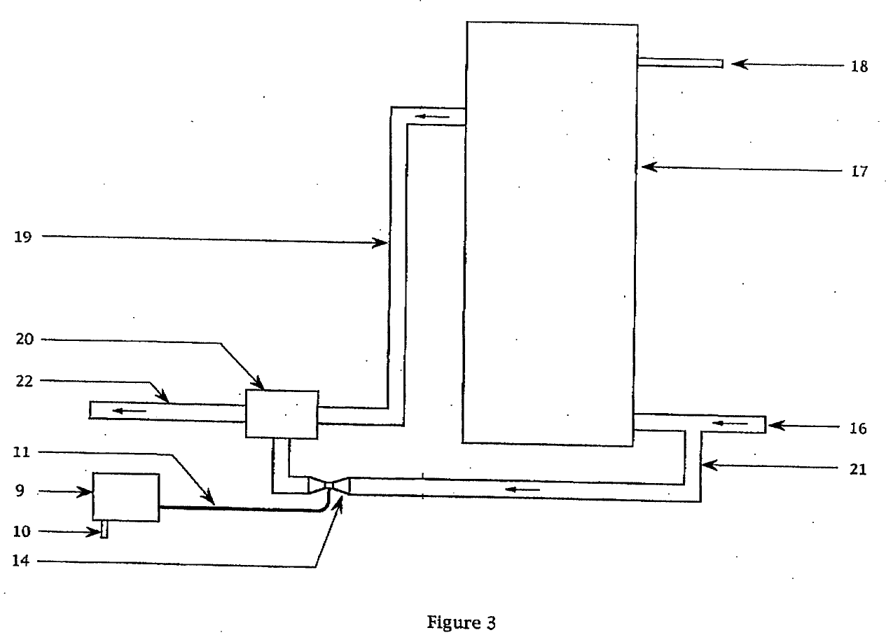

FIG. 4 is a compact form of the invention, including a diffuser,

FIG. 5 is a compact form of the invention, including a venturi,

FIG. 6 is an alternate form of the invention, including an oxygenator,

FIG. 7 is an alternate form of the invention, including a humidifier,

FIG. 8 is an alternate form of the invention, including a degasser,

FIG. 9 is an alternate form of the invention, including a friction tube and water filter.

### DESCRIPTION OF THE PREFERRED EMBODIMENT

The HVAC Water Purifier creates strong oxidants from air. These oxidants are created by using electrical energy, such as by passing air which may contain water vapour through a corona discharge field. The oxidants which are created may include one or more of the following: ozone (triatomic oxygen), hydroxyl radical, hydroxyl ion, hydrogen peroxide, atomic oxygen, atomic oxygen ion, diatomic oxygen ion, hydrogen ions, nitrogen Ions and similar. These oxidants are then dissolved into water by using a contact mechanism such as a porous diffuser or a venturi This mixture of “oxidants in water” or “oxidised water” then flows through the HVAC system. In the HVAC system itself, there may be a further phenomenon where ozone reacts with intermediary oxidants such as hydrogen peroxide and this creates further hydroxyl radicals.

The oxidised water may include:

i. Some oxidants which are properly dissolved in the water and are effectively in the liquid phase.

ii. Some oxidants which are not dissolved and are still in the gas phase, and for example may be seen as bubbles in the water which vent from the water surface. Alternatively the bubbles can be removed and reinjected before exiting the product.

iii. Some residual air (diatomic oxygen molecules and nitrogen molecules and water vapour) which are present because the efficiencies of the air preparation devices are less than 100%.

The oxidised water both cleans and purifies pollutants from the HVAC system:

i. Cleaning is primarily by a process of oxidation of inorganic and non-living organic substances in the water and on the HVAC system surfaces, also by a process of killing microorganisms which act as a substrate for other pollutants on the surfaces, and also by a process of friction where the oxidised water flows past the surfaces.

ii. Cleaning is also by a process of micro-flocculating salts in the water and then capturing the flocculant in a water filter.

iii. Purification is by a process of the oxidants causing denaturing of the protein structure in micro-organisms and thereby killing them.

Typically the Water Purifier Is operating whenever the HVAC system is operating. Therefore the water treatment is continuous or semi continuous, rather than periodic.

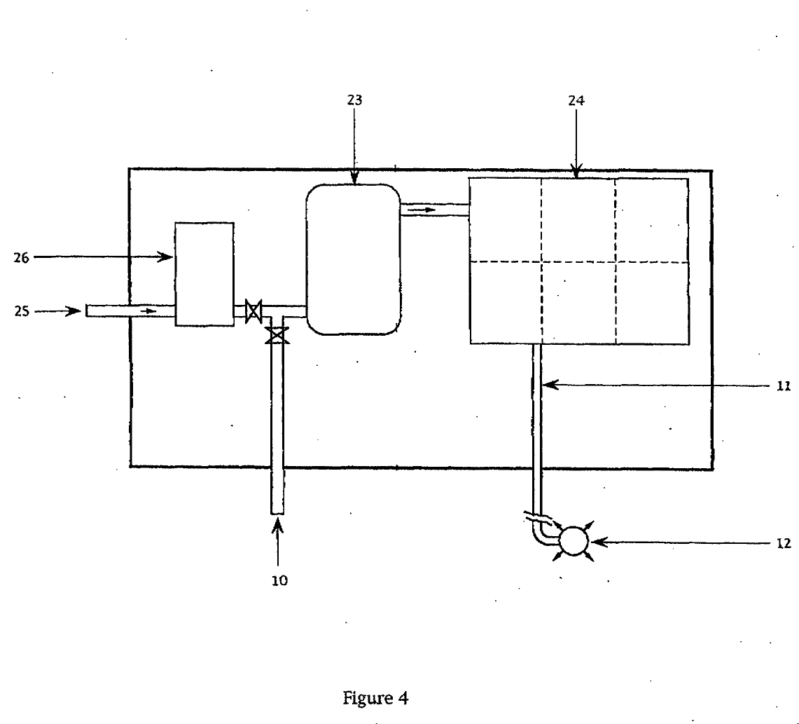

FIG. 4 shows a first embodiment of the invention, which is a compact Water Purifier to suit a residential evaporative cooler. The air feedstock may be supplied from the ambient air through air inlet 10 and flow directly to a compressor 23 and then to an oxidising chamber 24. Ambient air contains some natural water vapour. Alternatively the air feedstock may be supplied from ambient air through alternative air inlet 25 and then pass through a dryer 26 so as to remove the water vapour before it passes to the compressor and oxidising chamber. The oxidising chamber utilises a corona discharge field to create strong oxidants. The oxidants are in the gas phase and may also be in the aqueous phase as a vapour or liquid aerosol, They then pass through a tube 11 to a contactor 12. The contactor is preferably a porous diffuser which is remotely positioned in the basin of the evaporative cooler.

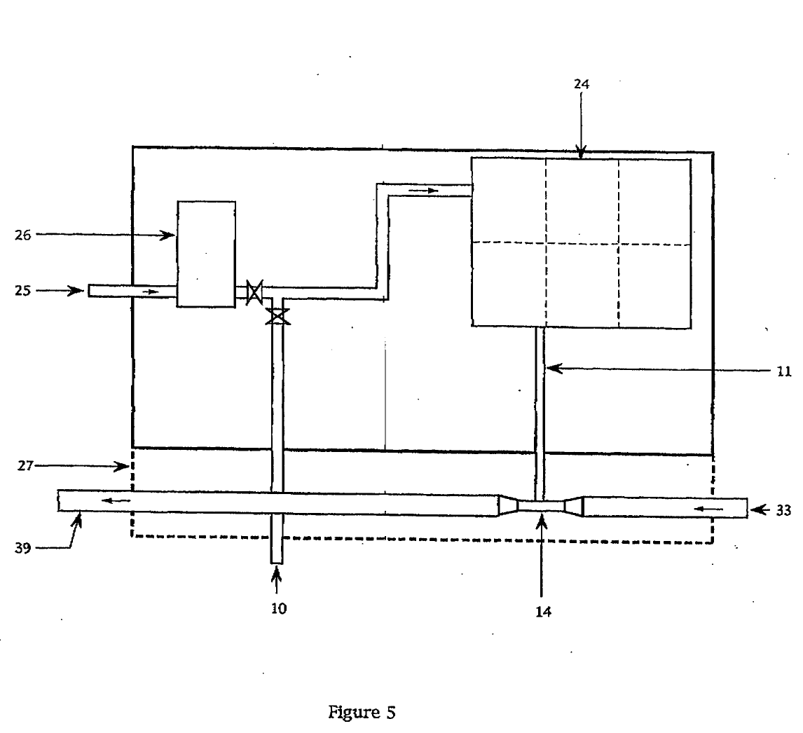

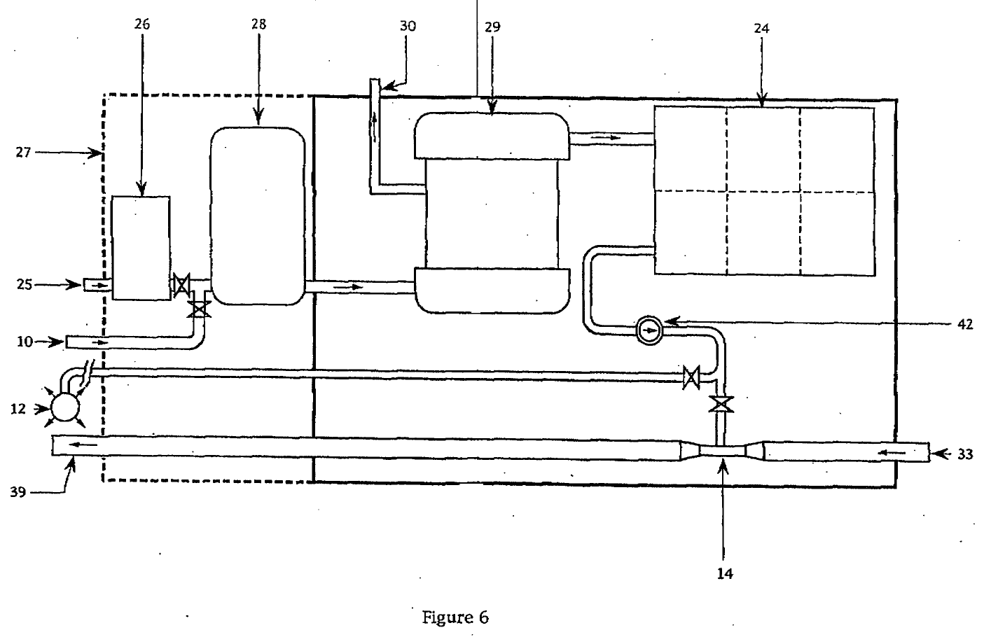

FIG. 5 shows a further form of the invention, which is also a compact Water Purifier to suit a residential evaporative cooler or a residential Hot/Warm Water System. The apparatus is similar to FIG. 4 but the contactor is a venturi injector 14. The evaporative cooler's existing recirculation pump is then used to provide the motive force. As the water flows through the venturi, a vacuum is created in the gas port of the venturi, which sucks gas through the oxidising chamber 24. Thus the compressor component is not required in one version of the invention, the parts shown in FIG. 5 are fully encapsulated in a potted mass. The dashed line 27 illustrates that the venturi can either be included in this potted mass or can be located remotely, The dashed line has a similar meaning in FIGS. 6 to 8.

FIG. 6 shows a further form of the invention to increase the concentration of oxidants, by using an oxygen concentrator device, also known as an oxygenator. The product, is a larger unit to suit Cooling Towers or commercial Hot/Warm Water Systems or large commercial Evaporative Coolers. The air feedstock may be supplied from the ambient air through inlet 10 and then passes through an air preparation system, such as a compressor 28 through a tube to an oxygenator 29 to remove nitrogen 30 and achieve a high oxygen concentration. Alternatively the air feedstock may be supplied from ambient air through alternative air inlet 25 and then pass through a dryer 26 before it passes to the compressor 28. The output from the oxygenator passes to the oxidising chamber 24. The remainder of the Water Purifier system is then as was previously described in reference to FIGS. 4 and 5. The contactor device can be a porous diffuser 12 or alternately be a venturi 14,

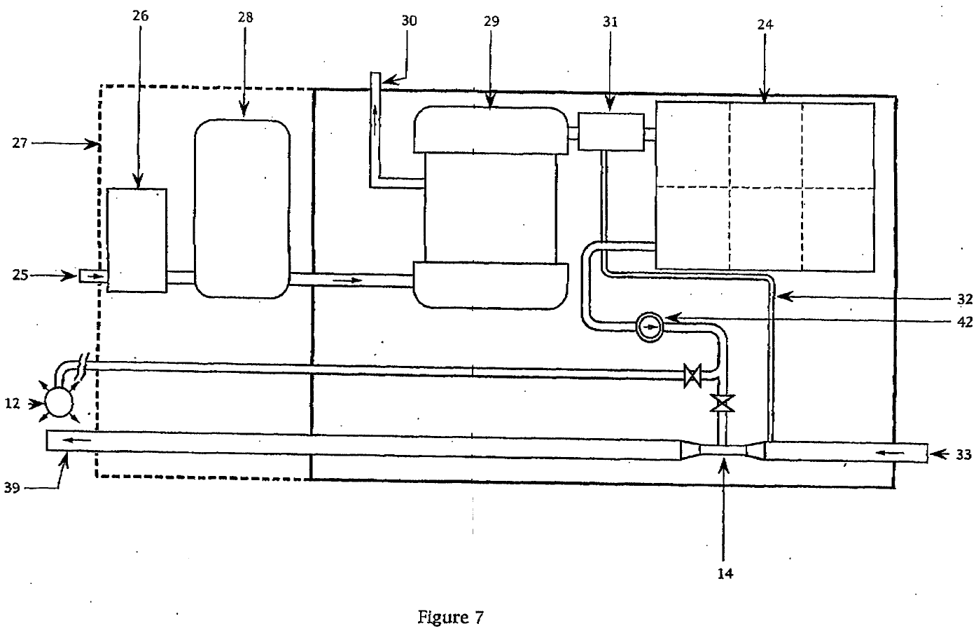

FIG. 7 shows a further form of the invention to create high concentrations of oxidants in the oxidant outlet, and also to optimise the efficiency and life of the product. The feedstock air enters the air inlet 25 and is dried 26 before being compressed 28 and oxygenated 29. The gas therefore mainly comprises dry oxygen. However before this gas flows Into the oxidation chamber 24 it is humidified by a humidifier device 31. A flow of water is bled from the main water flow through line 32 to the humidifier device 31 which mixes water in aerosol or droplet or vapour form into the gas which is flowing from the oxygenator to the oxidation chamber. The humidifier device preferably comprises a membrane contact device, which allows pressurised water to pass through small pores of a membrane and thus enter a flow of oxygen. Thus water vapour or aerosol (H2O) and oxygen (O2) and a minor quantity of residual air pass into the oxidation chamber. The remainder of the Water Purifier system is then as was previously described in reference to FIGS. 4 and 5. The contactor device can be a porous diffuser 12 or alternately be a venturi 14.

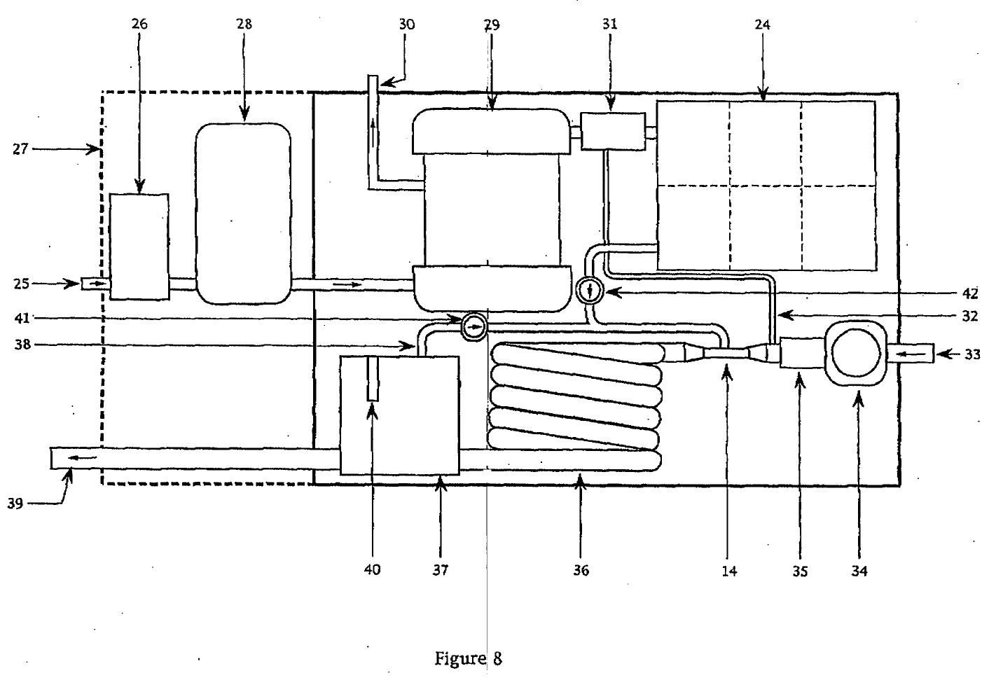

FIG. 8 shows a further form of the invention which includes efficient mixing, degassing and re-injection. The main water flow enters through water inlet 33 and then passes through water solenoid valve 34. The water feedstock may be from a pressurised mains system or it may be from a tank or keg or dam in which case a water pump may be included with the Water Purifier product. The optional solenoid 34 can serve both as a backflow prevention device or as an automatic method of activating the HVAC Water Purifier's electrics when connected to a flow switch 35, from which it receives an electrical signal. After the oxidants have been contacted with the water, they are mixed in the water by the mixing coil 36.

The oxidised water in the mixing coil contains some oxidants which are dissolved and some which are still in the form of bubbles. This “undissolved” component would normally be wasted and would vent to air at the first opportunity. In the invention the oxidised water then passes through a degasser chamber 37 where the bubbles are separated from the water. The bubbles are expelled as gas to the vent outlet 38, whilst the oxidised water leaves the product at the oxidised water outlet 39. One form of the degasser chamber comprises a pressure vessel or tank into which the oxidised water enters, and thus the water velocity slows and allows bubbles to rise to the surface of the water, which creates a gaseous space at the top of the chamber. As this gas builds up, the water level in the chamber reduces and a float switch 40 sends an electric signal to a degasser solenoid 41 which opens and allows the gas to vent through tube 38, until the float switch moves the solenoid back to the closed position. The vent tube 38 contains ozone gas. Preferably R is connected Into the gas line just downstream of check valve or solenoid 42, or connected to a second gas port on contactor 14, or into the gas port of a second contactor which may be positioned upstream or downstream of the first contactor. In this way the ozone gas is used efficiently.

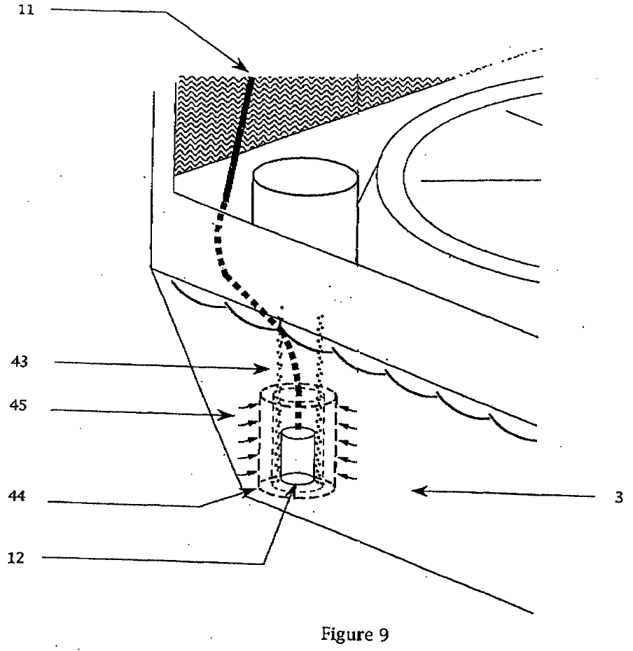

FIG. 9 shows an alternate variation of the invention where a special water filter and friction tube component is added to the HVAC Water Purifier. This component removes certain pollutants or substances from the water. In particular it can remove “flocculated salts” from the water.

The oxidants cause the salts to undergo a process of micro-flocculation as follows to change the salt component from a dissolved form to an un-dissolved form. The oxidants in the water cause organic pollutants in the water to become polar. These polar pollutants then bond with salts to create complex organic/inorganic compounds. These compounds flocculate from the water and can be captured in a filter. This process may be enhanced by using a cyclical timer to deliberately create partial oxidation by repeatedly turning the Water Purifier on and off, so that the oxidant concentrations in the water vary with time and it is ensured that for at least some of this time, the levels are such that partial oxidation occurs, as distinct from complete oxidation.

Preferably, a water filter of suitable micron size is placed in the water to filter the micro-flocculants out of the water, All that is then further required is a motive force to cause the water to pass through the filter. This can be a stand alone pump or it can be the existing main water pump In the HVAC system. Or the innovation described in the following point can be utilised, A friction tube may be used to cause the water to flow through this filter in a cost-effective manner, which does not require a further component to provide this motive force, as now described. The Water Purifier typically already includes an air compressor or air pump which delivers the oxidants through a tube 11 to a porous diffuser 12 located in the water basin 3. This porous diffuser emits gas bubbles 43 from its pores, some of which dissolve into the water and some of which rise quickly to the water surface. The diffuser 12 is located inside a cylindrical cartridge filter 44. In one variation of the invention, a friction tube or tubular shape may be further located on the inside of this cartridge filter. As the gas bubbles rise upwards out of the cartridge, a friction effect takes place which causes water 45 to be entrained into the filter. Thus water is sucked from the outside of the cartridge to the inside, at a low flow rate but on a continuous basis. Therefore there is an effective “water pump and water filter” in the HVAC system, which is cost effective because the motive force for the water movement is created by using a flow of pressurized gas already existing in the Water Purifier device

By changing or cleaning the water filter on a periodic basis, such as when the HVAC system receives a general service, it can be seen that the salts have been flocculated, filtered and then removed from the system entirely.

It has been proved by tests and investigation that the invention as described above can create two sets of oxidants depending upon the air inlets used.

i. In FIGS. 4 and 5, when air inlet 25 is used, the feedstock air is dried, water vapour is removed and oxygen and nitrogen remain. Thus the resultant feedstock does not contain hydrogen atoms. The main oxidant then created by the oxidising chamber is ozone in medium concentrations.

ii. In FIGS. 4 and 5, when air inlet 10 is used, the feedstock air contains water vapour, oxygen and nitrogen. The main oxidants created by the oxidising chamber are ozone in the gas phase in medium concentrations, and also hydrogen peroxide in the aqueous phase and hydroxyl radicals, both in significant concentrations.

iii. In FIG. 6, when air inlet 25 is used water vapour is removed by the dryer, nitrogen is removed by the oxygenator, and high concentrations of oxygen remain. The main oxidant then created by the oxidising chamber is ozone in high concentrations.

iv. In FIG. 6, when air inlet 10 is used, nitrogen is removed but water vapour and high concentrations of oxygen remain. The main oxidants created by the oxidising chamber are ozone in the gas phase in high concentrations, and hydrogen peroxide in the aqueous phase and hydroxyl radicals, both in medium concentrations.

V. In FIG. 7, air inlet 25 is used. The dryer removes water vapour, the oxygenator removes nitrogen and high concentrations of oxygen remain downstream of the oxygenator 29. The humidifier 31 then adds water vapour in a fine aerosol form. The main oxidants created by the oxidising chamber are ozone in the gas phase in high concentrations, and hydrogen peroxide in the aqueous phase and hydroxyls, both in high concentrations.

The advantage of creating hydroxyl radicals is that they are very strong oxidants and provide an advanced oxidation process. For example, measured in volts, the oxidation potential of chlorine gas is 1.36, ozone is 2.07 and the hydroxyl radical is 2.80. There are many substances, including some synthetic and natural organic chemicals, which have a slow reaction rate with ozone but a fast reaction rate with hydroxyls, and in such instances hydroxyls are superior oxidants. Hydroxyls have a short half life, being a fraction of a second whilst ozone has a longer half life, being up to 30 minutes in clean water. Therefore for micro-organism disinfection, where a residual oxidant level is required for a period of time, ozone is a superior oxidant. Other examples also exist where either hydroxyls or ozone or both, can be chosen to provide the optimum oxidant regime.

Further, the invention is able to create hydroxyl radicals in the downstream flow of water. An FIG. 7 for example, wet oxygen is used as feedstock to the oxidising chamber which creates ozone in the gas phase and hydrogen peroxide in the aqueous phase, and also creates some hydroxyl radicals. The ozone and the hydrogen peroxide are created independently from each other and at the same time and in a single operation, whilst the feedstock is passing through the discharge gap in the emitter. The ozone and hydrogen peroxide are then mixed into the main water flow at the contactor. The hydrogen peroxide then acts as an intermediary. It gradually reacts with some of the ozone, in this downstream flow, to create further hydroxyl radicals. Thus the invention provides ozone and hydroxyl radicals which are created or generated in the downstream flow, such as in the basin of the Evaporative Cooler or Cooling Tower or in the distribution pipework of the Hot/Warm Water System. If the hydroxyls were only created in the oxidising chamber itself, then they would not be able to do useful work in downstream water flow, as they would disappear quickly due to their short half life which is a fraction of a second, But because the invention allows them to be generated in a downstream flow, this limitation is solved, and the oxidants can act upon a larger body of water and also upon surfaces of the HVAC equipment.

Ozone decomposes in water with a natural half-life. When it does so, hydroxyl radicals are generated as a transient by-product. However the process described above, involving hydrogen peroxide, is a separate phenomenon and involves the generation of larger quantities of hydroxyl radicals from a reaction between ozone and hydrogen peroxide.

The presence of water vapour in the discharge space of the oxidising chamber acts to reduce the ozone output rate and the ozone concentration which would otherwise be achieved if the space was dry. However this effect is counteracted by the formation of hydrogen peroxide which in turn enables the generation of larger quantities of hydroxyl radicals.

The oxidising chamber is designed so that it can create ozone and hydrogen peroxide and hydroxyls, by receiving wet (humid) air or wet (humid) oxygen. A corona discharge field is developed. Mains electrical input is transformed into the optimum combination of voltage, frequency and wave shape, so as to disassociate the diatomic oxygen and water vapour molecules into atomic oxygen and hydrogen, to then enable recombination into the required oxidants.

The invention is designed to minimise corrosion rates and to extend component life, for applications where hydroxyl radicals and ozone are required and therefore wet air or wet oxygen feedstock is used.

i. In FIGS. 4 and 5, when inlet 10 is used, water vapour and nitrogen flow through the corona field in the oxidising chamber. Trace levels of substances may form, such as nitric acid, which may gradually corrode the surfaces of components in the oxidising chamber which are in the gas stream, including stainless steels. The oxidising chamber is designed so that it is non-corrosive, The oxidising chamber comprises emitters, power sources, printed circuit boards, etc. There may be multiple emitters, in parallel or in series, so as to achieve the desired oxidant output and concentrations. A corona field is created in the emitter and the feedstock flows through this field. The emitters include a high voltage electrode, an earthed electrode and a dielectric. The electrodes may be made of metals including stainless steels or other materials which are electrically conductive and such materials are corrosive to some extent. The dielectric is made of silicon or mica or ceramic based materials, including glass, which have high corrosion resistance. The emitter design is laminated so that the dielectric lies on top of the high voltage electrode, or the high voltage electrode is encapsulated in a dielectric. Therefore this electrode is not adjacent to the feedstock flow and thus it does not corrode. In addition, or alternatively, the earthed electrode can also be laminated by positioning a second dielectric against it, or it can be encapsulated by the dielectric. Thus one or both electrodes can be completely removed from the feedstock flowing through the emitter and thus corrosion is reduced and the efficiency of the oxidising chamber is maintained.

ii. In FIG. 6 there is an oxygenator which removes nitrogen and thus substances such as nitric acid do not form in the oxidising chamber and thus corrosion is controlled. However, in the case of inlet 10, the water vapour flows through the oxygenator which can damage the molecular sieve media in it and reduce media life or reduce the efficiency of oxygen concentration. The oxygenator is designed so that it includes an excessive amount of molecular sieve media, and where this media may be easily replaced at a regular service interval.

iii. FIG. 7 shows a preferred configuration of the HVAC Water Purifier. The dryer 26 removes water vapour so that the molecular sieve material in the oxygenator 29 is not damaged and so that oxygen concentration efficiency is maintained. The oxygenator removes nitrogen so that substances such as nitric acid do not form in the oxidising chamber. The water vapour is added to the system at the optimum location, namely the humidifier 31, so that hydroxyl radicals can be created either in the oxidising chamber itself or in downstream lines via the hydrogen peroxide intermediary. The oxidising chamber can also utilise an emitter design with laminated electrodes as previously described, so as to provide an extra level of corrosion protection.

The invention may be configured by using various component options and configurations, including:

A timer device may be connected to cause the device to cycle on and off. In one variation of the invention, this cycling operation can make an important contribution towards achievement of the benefits obtained, by achieving the partial oxidation of organic pollutants which then combine with salts and result in a process known as micro-flocculation. The dryer component 26 may comprise desiccant media, with or without a regenerative heater circuit, or may be a refrigerative dryer, or may be a coelescer or water trap device or mist filter. A particulate filter may be added to remove pollutants to protect the compressor and oxygenator and oxidising chamber. The oxygenator may utilise a molecular sieve, or pressure swing absorption design, or membrane design. The compressor may be a rotary or reciprocating device or an air pump or a diaphragm pump. The compressor 28 and oxygenator 29 may be replaced with bottled oxygen. The humidifier may utilise a porous membrane or any other method which allows the oxygen to become partially or fully saturated with water at up to 100% relative humidity. The oxidising chamber may comprise corona discharge, plasma discharge, silent electrical discharge, dielectric barrier AC discharge or ultra-violet radiation or other electrical methods of creating oxidants. The oxidising chamber may include a catalyst such as Titanium Dioxide, with or without electrical potential applied to the catalyst surfaces. The emitters in the oxidising chamber may comprises electrodes which are tubular in shape or which utilise a parallel plate shape. The electrodes may be solid material or may be granular. The contactor may comprise a venturi, or a porous diffuser which bubbles into a basin or contact tower or pipe, or a membrane device. Or the contactor may utilise a peristaltic pump through which the oxidised gas passes so that this pump forces the gas through a porous diffuser into the water flow. Or if mains water pressure is not used, then a dual head peristaltic pump may be utilised, where one pump head creates pressurised water for the purpose of the main water flow and the other head creates pressurised oxidised gas which is then forced through a porous diffuser into the water flow. The mixing coil may be replaced by or used in conjunction with a static mixing device placed in a section of pipe. The product can be configured with or without the alternative air inlets previously described, and preferably would only Incorporate the inlets which result in hydroxyl radicals and ozone being created, including hydrogen peroxide as an intermediary, rather than ozone alone. The oxidising chamber may include multiple emitters and these emitters are preferably each encapsulated in a potting compound such as epoxy. This provides a method of achieving low electrical magnetic interference, safe electrical insulation and waterproofing. Cooling fins may be moulded into the cast potted shape.

The advantages of the Invention include the following:

i. Hydroxyl radicals and ozone are created in the downstream water flow in the HVAC system. Thus these oxidants can do useful work such as cleaning and purifying the main body of water and the surfaces of the HVAC system. The hydroxyl radicals are created in the HVAC system itself, due to a reaction between the ozone and intermediary oxidants such as hydrogen peroxide, which are previously created in the oxidising chamber of the product and then mixed into the main water flow. The hydroxyl radicals are very strong oxidants which are ideal for oxidising inorganics and non-living organics whilst the ozone creates a temporary residual oxidation level which is ideal for killing micro-organisms.

ii. The process is an all-electric advanced oxidation process. There are no chemicals or consumables. This creates significant on-going purchasing and logistics savings. The combination of this all-electric process together with hydroxyls being generated downstream in a line (as per point i above), is a unique and innovative combination.

iii. Scale build up on surfaces (also called scale or lime or bio-film) is reduced. This includes mineral scale and organic film. This can reduce fouling of moving parts and can reduce corrosion. This increases the life of components, reduces the need for servicing, reduces failure rates, reduces aesthetic problems such as the formation of white stains, etc. It can also increase the cooling efficiency of pads, and reduce electricity usage.

iv. There are no hazardous chemicals. This creates occupational health and safety advantages, and logistics advantages during transport, storage and handling.

V. The oxidants clean efficiently and purify efficiently. A wide range of micro-organisms are killed, including Legionella bacteria and Giardia and Cryptospordium protozoa.

vi. Unpleasant odours are reduced. This is advantageous for occupants as well as neighbouring residents.

vii. The colour of the water improves and becomes clearer. This is an advantage when occupants site the water in an Evaporative Cooler, or in the case of bleed water which leaves the Cooler. It is an obvious advantage in the case of ingested water.

viii, Water usage reduces which is “environmentally friendly” as it contributes towards water conservation and reduces waste water. This occurs because purge water can be reduced or eliminated entirely.

ix. Running costs can be reduced due to lower water usage, and reduced regular maintenance. This is due to purge water being reduced or eliminated entirely, and also due to less scale build-up and corrosion.

x. The oxidants do not excessively corrode the HVAC system fittings. Corrosion rates can be less than occur in the case of chlorinated mains water,

xi. Instruments can be used to give a sufficiently precise indication of whether the oxidation process is taking place. One method is to use a redox or ORP meter, also known as an oxidation reduction potential meter,

Thus it can be seen that the quality of water and surfaces in HVAC systems, and resultant air quality, can be effectively controlled, and can be continuously cleaned and purified without the use of chemicals or ultraviolet radiation or without reliance on chlorine in mains water. By connecting a unit which provides an advanced oxidation process and passing the oxidised water through the HVAC system, an effective and safe system of cleaning and purification is provided. In addition the invention can be applied to any equipment using recirculating water to cool air, or air to cool water, or other fluid heat transfer, in residential or commercial or industrial processes.

Although alternate forms of the invention have been described in some detail it is to be realised the invention is not to be limited thereto but can include variations and modifications falling within the spirit and scope of the invention.

1. A method of cleaning and purifying water or surfaces or air in HVAC systems (heating, ventilating or air conditioning systems), including the steps of producing oxidants in a fluid (including air and/or water) which is a part of the HVAC system to react with and remove contaminants in the HVAC system, where these oxidants are generated in a single oxidising chamber from molecules of air and/or water and thus contain oxygen and/or hydrogen atoms, and include ozone together with one or more other oxidants, such as hydroxyl radicals or hydrogen peroxide.

2. A method of cleaning and purifying water or air or surfaces in HVAC systems, including the steps of providing a flow of fluid as a part of the HVAC system, passing air which contains oxygen and water vapour through a single oxidising chamber to produce oxidants in the form of hydrogen peroxide and one or more of hydroxyl radicals, ozone, hydroxyl ions, atomic oxygen and atomic oxygen ions and injecting and mixing be oxidants in a flow of fluid within the HVAC system.

3. A method of cleaning and purifying water or surfaces or air in HVAC systems wherein ozone and hydrogen peroxide are produced in a single oxidising chamber and then injected into water wherein the hydrogen peroxide then acts as an intermediary and reacts with the ozone to form hydroxyl radicals downstream of the point of injection into the flow of fluid, including in the HVAC system through which the oxidised fluid flows.

4. A method of cleaning and purifying water or surfaces or air in HVAC systems as defined in any one of claims 1, 2 or 3 including the step of generating the oxidants by an electrical means only.

5. A method of cleaning and purifying water or surfaces or air in HVAC systems as defined in any one of claims 1, 2 or 3, including the steps of passing air through an ozone generator, then injecting and mixing the ozone into the fluid flowing through the HVAC system in order to clean and purify it.

6. A method of cleaning and purifying water or surfaces or air in HVAC systems as defined in any one of claims 1, 2 or 3, including the steps of drying and compressing air, passing the dried compressed air through an oxygenator to remove nitrogen from the air, adding water in the form of aerosol or vapour or mist or droplets into the gas, passing this gas which has high concentrations of oxygen and water vapour through an electrical oxidising chamber and injecting the resultant oxidants into the flow of fluid.

7. Apparatus for cleaning and purifying HVAC systems by the method as defined in any one of claims 1, 2 or 3, said apparatus including an air inlet, an oxidant or ozone generator having an inlet connected to the air inlet, and an outlet connected to a passage between the fluid inlet and outlet whereby the products from the oxidant or ozone generator are passed into and mixed with the fluid to clean and purify the HVAC system.

8. Apparatus as defined in claim 7 characterised by an oxygenator positioned in the air line prior to the oxidant or ozone generator whereby oxygen enriched air is passed to the oxidant or ozone generator to produce ozone and/or hydroxyl radicals generated down stream in the fluid flow.

9. An apparatus as defined in claim 7 or claim 8 characterised by an air drier positioned in the air inlet line.

10. An apparatus as defined in any one of claims 7 to 9 characterised by an air compressor to pressurise the inlet air.

11. An apparatus as defined in any in of claims 7 to 10 characterised by a humidifier positioned in the gas line between the oxygenator and the oxidant or ozone generator to humidify the gas by water spray, water aerosol, mist, droplet or steam.

12. An apparatus as defined in any one of claims 7 to 11 characterised in that the oxidised fluid flow passes through a mixer prior to entering the HVAC system.

13. An apparatus as defined in any one of claims 7 to 12 characterised by passing oxidised water flow through a degasser to remove undissolved gasses and to reinject those gases into the water flow prior to exiting the apparatus and entering the HVAC system.

14. Apparatus for cleaning and purifying HVAC systems as defined in any one of claims 7 to 13, said apparatus including means of micro-flocculating salts in the water, producing a motive force in the water by bubbling air through a friction tube in the water, and passing this flocculated material and water through a water filter, thus reducing the concentration of the salts in the water.

15. Apparatus for cleaning and purifying HVAC systems as defined in any one of claims 7 to 14, said apparatus including emitters which create a corona discharge or similar field, where the emitters include one or more conductive electrodes which are encapsulated or laminated by dielectric material so that the electrodes are not exposed or adjacent to the gas flow.

16. A method of cleaning and purifying water or surfaces or air in HVAC systems or an apparatus as defined in any one of claims 1 to 15 characterised in that the HVAC systems are selected from systems including evaporative coolers, cooling towers, hot or warm water systems, spa pools or swimming pools, ducted heaters and ducted air conditioners.

17. A method of cleaning or purifying water or surfaces or air in HVAC systems as defined in any one of claims 1 to 6 or an apparatus as defined in any one of claims 7 to 16 which includes multiple oxidising chambers situated in parallel to increase the product flow.
